(19) United States
(12) Patent Application Publication
(10) Pub. No.: US 2022/0165419 A1
(43) Pub. Date: May 26, 2022

(54) METHODS FOR PREDICTING MEDICATION INDUCED RESPIRATORY DEPRESSION

(71) Applicant: CERNER INNOVATION, INC., Kansas City, KS (US)

(72) Inventors: Michelle R. Padgett, Leawood, KS (US); Leslie A. Lindsey, Liberty, MO (US); Andrew C. Lueders, Kansas City, MO (US); Maria Fernandez-Martin, Overland Park, KS (US); Jaleesa Amezaga, Torrance, CA (US); Maria Caldera, Torrance, CA (US); Tammy Ginder, Torrance, CA (US); Lindy Lipscomb, Torrance, CA (US); Julie Maglanoc, Torrance, CA (US); Golda Miclat, Torrance, CA (US); Pamela Michael, Torrance, CA (US); Joanne Mulvaney, Torrance, CA (US); Daniel Palma, Torrance, CA (US); Felix Pham, Torrance, CA (US); Clare Torres, Torrance, CA (US); Wendy Waldman, Torrance, CA (US)

(21) Appl. No.: 17/530,613

(22) Filed: Nov. 19, 2021

Related U.S. Application Data
(60) Provisional application No. 63/116,507, filed on Nov. 20, 2020.

(51) Int. Cl. G16H 50/20; G16H 70/40; G16H 20/10; G16H 10/60

(52) U.S. Cl. CPC G16H 50/20; G16H 10/60; G16H 20/10; G16H 70/40

(57) ABSTRACT

Methods, systems, and computer-readable media are disclosed herein for predicting medication induced respiratory depression in patients. Herein, when indications are received for particular events being logged to a computerized clinical workflow, a query of patient's clinical record(s) is preformed to locate predetermined factors that are associated with medication induced respiratory depression. An overall value is calculated to stratify the possibility of adverse respiratory events for the patient. Warnings, detailed intervention instructions, and complete medical orders can be automatically generated and provided to a clinician and/or automatically input to the clinical workflow of the patient for performance.

### CROSS REFERENCE TO RELATED APPLICATIONS

The present application is a nonprovisional that claims the benefit of and priority to U.S. Provisional Application No. 63/116,507, filed on Nov. 20, 2020, the entirety of which is herein incorporated by reference.

### BACKGROUND

Clinical workflows are used to electronically document a clinician's entry of medical orders, administrations, and to coordinate patient treatments in a computerized system.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

One aspect of the present disclosure relates to a method for predicting medication induced respiratory depression. In aspects, the method is performed and/or repeated for each received indication of an admission event, a diagnosis event, a clinical event, and/or an order event corresponding to a patient. An electronic record corresponding to the patient is queried for a presence of one or more of a plurality of predefined data elements, in some aspects. Based on the presence of the one or more of the plurality of predefined data elements, a total element value of the patient is automatically determined, in aspects. In some aspects, each of the one or more of the plurality of predefined data elements has an assigned corresponding value. The total element value is determined to meet a threshold, in an aspect. When the total element value is determined to meet the threshold, a notice is automatically generated and presented, for example, to a user or clinician. The notice includes the total element value and an instruction that is based on the total element value, in various aspects.

In another aspect, the present disclosure relates to a non-transitory computer-readable storage medium having instructions embodied thereon, the instructions being executable by one or more processors to perform a method for predicting medication induced respiratory depression. In aspects, the method can be performed and/or repeated for each instance of a received indication of an admission event, a diagnosis event, a clinical event, and/or an order event corresponding to a patient. An electronic record corresponding to the patient is queried for a presence of one or more of a plurality of predefined data elements, in some aspects. Based on the presence of the one or more of the plurality of predefined data elements, a total element value of the patient is automatically determined, in an aspect. In aspects, each of the one or more of the plurality of predefined data elements has an assigned corresponding value. The total element value is determined to meet a threshold, in some aspects. When the total element value is determined to meet the threshold, in an aspect, a notice is automatically generated. Further, the notice is automatically presented, for example, to a user or clinician. The notice includes the total element value and an instruction that is based on the total element value.

In yet another aspect, the present disclosure relates to a non-transitory computer-readable storage medium having instructions embodied thereon, the instructions being executable by one or more processors to perform a method for predicting medication induced respiratory depression. For each instance of receipt of an indication of an admission event, a diagnosis event, a clinical event, and/or an order event corresponding to a patient, an electronic record corresponding to the patient is queried for the presence of one or more of a plurality of predefined data elements, in aspects. Based on the presence of the one or more of the plurality of predefined data elements, a total element value of the patient is automatically determined, in some aspects. Each of the one or more plurality of predefined data elements has an assigned corresponding value, for example. As such, the total element value can be determined to meet a first, second, or third threshold, in some aspects. When the total element value is determined to meet the first, second, or third threshold, in various aspects, a notice is automatically generated and presented, for example, to a user or clinician. The notice, in some aspects, can include the total element value and a particular recommended intervention. In aspects, the particular recommended intervention can be based on one of the first, second, or third threshold that is determined to be met.

### BRIEF DESCRIPTION OF THE DRAWINGS

Illustrative aspects of the present invention are described in detail below with reference to the attached drawing figures, and wherein:

FIG. 1 illustrates a computing environment, in accordance with aspects;

FIG. 2 depicts a method for predicting medication induced respiratory depression, in accordance with aspects;

FIG. 3 depicts one or more software and/or hardware components, in accordance with aspects;

FIG. 4 depicts another method for predicting medication induced respiratory depression, in accordance with aspects;

FIG. 5 illustrates a flowchart of an example implementation of a method for predicting medication induced respiratory depression, in accordance with aspects;

FIGS. 6 and 7 depict example graphical user interfaces for displaying notices for predicted medication induced respiratory depression, in accordance with aspects;

FIG. 8 depicts an example graphical user interface for displaying notices for predicted medication induced respiratory depression, in accordance with aspects;

FIG. 9 depicts an example graphical user interface for displaying notices for predicted medication induced respiratory depression, in accordance with aspects;

FIG. 10 depicts an example graphical user interface for displaying notices for predicted medication induced respiratory depression, in accordance with aspects; and

FIGS. 11-13 depict tables listing examples of diagnosis or medical codes that correspond to data elements, in accordance with aspects.

### DETAILED DESCRIPTION

The subject matter of the present invention is being described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Terms should not be interpreted as implying any particular order among or between various steps herein disclosed unless and except when the order of individual steps is explicitly described. As such, although the terms “step” and/or “block” can be used herein to connote different elements of system and/or methods, the terms should not be interpreted as implying any particular order and/or dependencies among or between various components and/or steps herein disclosed unless and except when the order of individual steps is explicitly described. The present disclosure will now be described more fully herein with reference to the accompanying drawings, which may not be drawn to scale and which are not to be construed as limiting. Indeed, the present invention can be embodied in many different forms and should not be construed as limited to the aspects set forth herein. Further, it will be apparent from this Detailed Description that the technological solutions disclosed herein are only a portion of those provided by the present invention. As such, the technological problems, solutions, advances, and improvements expressly referenced and explained herein should not be construed in a way that would limit the benefits, improvements, and/or practical application of the discussed aspects of the present invention.

Aspects herein provide computer-readable media and methods for predicting medication induced respiratory depression in a real-time clinical workflow and automatically generating and inputting orders that are responsive to risk determinations within the clinical workflow. Conventional computerized clinical workflows do not autonomously predict the possibility of medication induced respiratory depression occurring for patients. In contrast, aspects herein provide an improvement over conventional computerized clinical workflows by adding a new functionality that includes autonomously and dynamically making predictions of medication induced respiratory depression occurring in patients, wherein the assessment and predication are performed in near real-time with electronic documentation and electronic events occurring or being input to the clinical workflow. For example, aspects herein can predict the future potential for a patient to experience an adverse medical event such as respiratory depression, in real-time or near real-time, wherein predictions, determinations, and/or evaluations of future potential for respiratory depression are automatically triggered by, responsive to, and/or performed within the course of clinical treatment, based on indications received in the clinical workflow for an admission, a medical order for drugs or treatment, documentation of the administration of medication(s), laboratory results, and the like. The real-time predictions are performed in an on-going manner throughout the computerized clinical workflow and up to the completion of a patient discharge (e.g., an indication is received of a patient discharge event being logged or input to the clinical workflow). Thus, aspects herein predict medication induced respiratory depression in a real-time computerized clinical workflow.

Further, aspects herein can automatically generate, display, and/or input notices and clinical intervention orders into the computerized clinical workflow, in response to the autonomously made predictions and other determinations. The ability to predict, create and send notices, and generate and input clinical intervention orders, can be implemented for a plurality of patients and across diverse clinical workflows (e.g., independent of the patient diagnosis, surgical treatment, demographics) such that aspects herein can be scaled up.

Aspects herein include media and methods for a computer tool that runs in the background of a computerized clinical workflow and which automatically determines medication induced respiratory depression potential for patients, without requiring manual or user-input requests. The computer tool intelligently self-triggers prediction assessments and determinations based on indications of specific workflow events occurring in the clinical workflow, and the self-triggered assessments and determinations are made in real-time or near real-time with the entry or occurrence of specific workflow events. Additionally, in some aspects, electronic orders for interventions that are responsive to the level of predicted medicine induced respiratory depression can be automatically created and automatically inserted into the computerized clinical workflow of a patient, again without requiring manual or user-input requests.

Accordingly, the dynamic, self-triggering computer tool integrates with a computerized clinical workflow to individually monitor, assess, and instantiate new recommended intervention orders within the computerized clinical workflow for a plurality of patients, in real-time or near real-time, which reduces the likelihood of adverse patient outcomes resulting from medication induced respiratory depression. In addition to being an improvement over conventional computerized clinical workflows, the computer tool provides a practical application of the technology. The computer tool addresses and solves the problem of patient information being stored in different data stores, different portions of large volume electronic records, being encoded in multiple, different data formats including non-human readable formats (e.g., metadata), human readable formats (e.g., HL7 messages), as structured data, and as unstructured data (e.g., images). The de-centralization and variety of electronic data for a patient creates a significant hurdle and impediment to performing, within a clinical workflow, a multi-factored prediction for medication induced respiratory depression for a single patient at all, much less for performance in real-time or near real-time during the patient's treatment. As such, the computer tool autonomously self-triggers real-time or near real-time predictions for medication induced respiratory depression occurring for a plurality of patients, across all types of clinical workflows, which is an unconventional solution to the limitations and problems found in conventional systems, which rely on de-centralized and varied electronic patient data that may not be readily searchable or accessible.

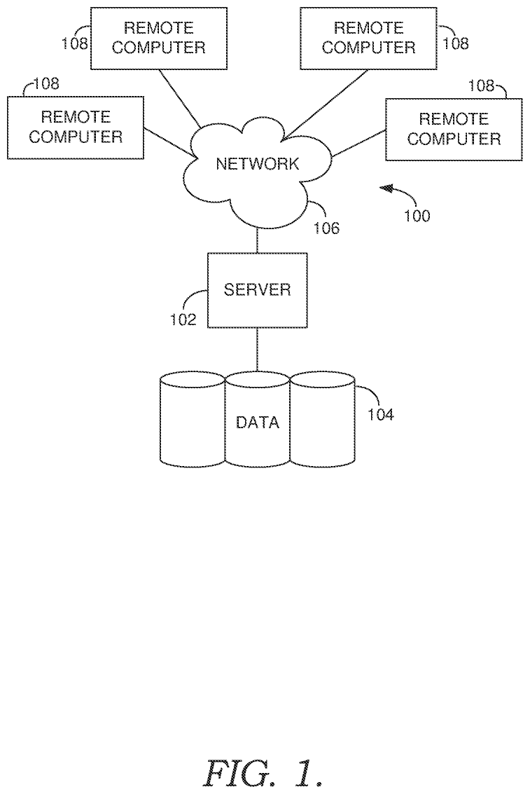

Beginning with FIG. 1, a computing environment 100 that is suitable for use in implementing aspects of the present invention is depicted. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. Generally, in aspects, the computing environment 100 is a medical-information computing-system environment. However, this is just one example and the computing environment 100 can be operational with other types, other kinds, or other-purpose computing system environments or configurations. Examples of computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

In aspects, the computing environment 100 can be described in the general context of computer instructions, such as program modules, applications, and/or extensions, being read and executed by a computing device. Examples of computer instructions can include routines, programs, objects, components, and/or data structures that perform particular tasks or implement particular abstract data types. The aspects discussed herein can be practiced in centralized and/or distributed computing environments, i.e., where computer tasks are performed utilizing remote processing devices that are linked through a communications network, whether hardwired, wireless, or a combination thereof. In a distributed configuration, computer instructions might be stored or located in association with one or more local and/or remote computer storage media (e.g., memory storage devices). Accordingly, different portions of computer instructions for implementing the computer tool in the computing environment 100 may be executed and run on different devices, whether local, remote, stationary, and/or mobile.

With continued reference to FIG. 1, the computing environment 100 comprises a computing device 102, shown in the example form of a server. Although illustrated as one component in FIG. 1, the present invention can utilize a plurality of local servers and/or remote servers in the computing environment 100. The computing device 102 can include components such as a processing unit, internal system memory, and a suitable system bus for coupling to various components, including electronic storage, memory, and the like, such as a data store, a database, and/or a database cluster. Example components of the computing device 102 include a processing unit, internal system memory, and a suitable system bus for coupling various components, including a data store 104, with the computing device 102. An example system bus might be any of several types of bus structures, including a memory bus or memory controller, a peripheral bus, and a local bus, using any of a variety of bus architectures. Examples of bus architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The computing device 102 includes or has access to a variety of non-transitory computer-readable media. Computer-readable media can be any available media that is locally and/or remotely accessible by the computing device 102, and includes volatile, nonvolatile, removable, and non-removable media. By way of example, and not limitation, computer-readable media can comprise computer storage media and communication media. Computer storage media includes volatile, nonvolatile, removable, and non-removable media, as implemented in any method or technology for storage of information, such as computer-readable instructions, data structures, program modules, or other data.

The computing device 102 can include or can have access to computer-readable media. Computer-readable media can be any available media that can be accessed by computing device 102, and includes volatile and nonvolatile media, as well as removable and non-removable media. By way of example, and not limitation, computer-readable media can include computer storage media and communication media.

Computer storage media can include, without limitation, volatile and nonvolatile media, as well as removable and non-removable media, implemented in any method or technology for storage of information, such as computer-readable instructions, data structures, program modules, or other data. In this regard, computer storage media can include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which can be accessed by the computing device 102. Computer storage media does not comprise signals per se.

Communication media typically embodies computer-readable instructions, data structures, program modules, or other data in a modulated data signal, such as a carrier wave or other transport mechanism, and can include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also can be included within the scope of computer-readable media.

The computing device 102 might operate in a network 106 using logical connections to one or more remote computers 108. In some aspects, the one or more remote computers 108 can be located at a variety of locations, such as medical facilities, research environments, and/or clinical laboratories (e.g., molecular diagnostic laboratories), as well as hospitals, other inpatient settings (e.g., surgical centers), veterinary environments, ambulatory settings, medical billing offices, financial offices, hospital administration settings, home healthcare environments, and/or clinicians' offices). As used herein, “clinicians,” “medical professionals,” or “healthcare providers” can include: physicians; specialists such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like.

In aspects, the computing device 102 uses logical connections to communicate with one or more remote computers 108 within the computing environment 100. In aspects where the network 106 includes a wireless network, the computing device 102 can employ a modem to establish communications with the Internet, the computing device 102 can connect to the Internet using Wi-Fi or wireless access points, or the server can use a wireless network adapter to access the Internet. The computing device 102 engages in two-way communication with any or all of the components and devices illustrated in FIG. 1, using the network 106. Accordingly, the computing device 102 can send data to and receive data from the remote computers 108 over the network 106.

The network 106 is a computer network that can include local area networks (LANs) and/or wide area networks (WANs), in some aspects. The network 106 can include wireless and/or physical (e.g., hardwired) connections. Examples of networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. When the network 106 includes a WAN-type configuration, the computing device 102 might comprise a modem or other means for establishing communications over the WAN, such as the Internet, in such aspects. As such, the network 106, can provide the components and devices access to the Internet and web-based applications.

The network 106 can include an entity-wide network, campus-wide network, an office-wide network, an enterprise-wide networks, and the Internet. In the network 106, applications, extensions, program modules or portions thereof might be stored in association with the computing device 102, the data store 104, and any of the one or more remote computers 108. For example, various application programs can reside on the memory associated with any one or more of the remote computers 108. In the computing environment 100, which is illustrated as being a distributed configuration of the network 106, the components and devices can communicate with one another and can be linked to each other using a network 106. It will be appreciated by those of ordinary skill in the art that the network connections shown are exemplary and other means of establishing a communications link between the computers (e.g., computing device 102 and remote computers 108) might be utilized.

In operation, an organization might enter commands and information into the computing device 102 or convey the commands and information, for example, directly in peer-to-peer or near-field communication, or through the network 106 using telecommunications or Wi-Fi, to the computing device 102 via one or more of the remote computers 108 through input devices, such as a keyboard, a pointing device (e.g., a mouse), a trackball, as stylus, or a touch pad. Other input devices comprise microphones, satellite dishes, scanners, or the like. Commands and information might also be sent directly from a remote healthcare device to the computing device 102. In addition to a screen, monitor, or touchscreen component, the computing device 102 and/or remote computers 108 might comprise other peripheral output devices, such as speakers and printers.

The computing environment 100 includes one or more remote computers 108, which may be accessed by the computing device 102 over the network 106 or directly using peer-to-peer connections or mesh networking, in various aspects. The remote computers 108 might be servers, routers, network personal computers, peer devices, network nodes, computing devices, personal digital assistants, personal mobile devices, medical devices, patient monitoring equipment, or the like, and might comprise some or all of the elements described above in relation to the computing device 102. The one or more remote computers 108 can include multiple computing devices, in various aspects. In aspects where the network 106 is distributed in configuration, the one or more remote computers 108 can be located at one or more different geographic locations. In an aspect where the one or more remote computers 108 are a plurality of computing devices, each of the plurality of computing devices can be located across various locations such as buildings in a campus, medical and research facilities at a medical complex, offices or “branches” of a banking/credit entity, or can be mobile devices that are wearable or carried by personnel, or attached to vehicles or trackable items in a warehouse, for example. In some aspects, the remote computers 108 are physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. The remote computers 108 might also be physically located in nontraditional healthcare environments so that the entire healthcare community might be capable of integration on the network 106. In other aspects, the remote computers 108 can be physically located in a non-medical setting, such as a packing and shipping facility or deployed within a fleet of delivery or courier vehicles.

Continuing, the computing environment 100 includes a data store 104. Although shown as a single component, the data store 104 can be implemented using multiple data stores that are communicatively coupled to one another, independent of the geographic or physical location of a memory device. The data store 104 can, for example, store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. The data store 104 can, for example, also store data in the form of electronic records, such as electronic medical records of patients, patient-specific documents and historical records, transaction records, billing records, task and workflow records, chronological event records, and the like. Generally, the data store 104 includes physical memory that is configured to store information encoded in data. For example, the data store 104 can provide storage for computer-readable instructions, computer-executable instructions, data structures, data arrays, computer programs, applications, and other data that supports the functions and actions to be undertaken using the computing environment 100 and components shown in the example of FIG. 1.

As shown in the example of FIG. 1, when the computing environment 100 operates with distributed components that are communicatively coupled via the network 106, computer instructions, applications, extensions, and/or program modules can be located in local and/or remote computer storage media (e.g., memory storage devices). Aspects of the present invention can be described in the context of computer-executable instructions, such as program modules, being executed by a computing device. Program modules can include, but are not limited to, routines, programs, objects, components, and data structures that perform particular tasks or implement particular abstract data types. In aspects, the computing device 102 can access, retrieve, communicate, receive, and update information stored in the data store 104, including program modules. Accordingly, the computing device 102 can execute, using a processor, computer instructions stored in the data store 104 in order to perform aspects described herein.

Although internal components of the devices in FIG. 1, such as the computing device 102, are not illustrated, those of ordinary skill in the art will appreciate that internal components and their interconnection are present in the devices of FIG. 1. Accordingly, additional details concerning the internal construction device are not further disclosed herein. Although many other internal components of the computing device 102 and the remote computers 108 are not shown, such components and their interconnection are known. Accordingly, additional details concerning the internal construction of the computing device 102 and the remote computers 108 are not further disclosed herein.

Additionally, it will be understood by those of ordinary skill in the art that the computing environment 100 is just one example of a suitable computing environment and is not intended to limit the scope of use or functionality of the present invention. Similarly, the computing environment 100 should not be interpreted as imputing any dependency and/or any requirements with regard to each component and combination(s) of components illustrated in FIG. 1. It will be appreciated by those having ordinary skill in the art that the connections illustrated in FIG. 1 are also examples as other methods, hardware, software, and devices for establishing a communications link between the components, devices, systems, and entities, as shown in FIG. 1, can be utilized in implementation of the present invention. Although the connections are depicted using one or more solid lines, it will be understood by those having ordinary skill in the art that the example connections of FIG. 1 can be hardwired or wireless, and can use intermediary components that have been omitted or not included in FIG. 1 for simplicity's sake. As such, the absence of components from FIG. 1 should be not be interpreted as limiting the present invention to exclude additional components and combination(s) of components. Moreover, though devices and components are represented in FIG. 1 as singular devices and components, it will be appreciated that some aspects can include a plurality of the devices and components such that FIG. 1 should not be considered as limiting the number of a device or component.

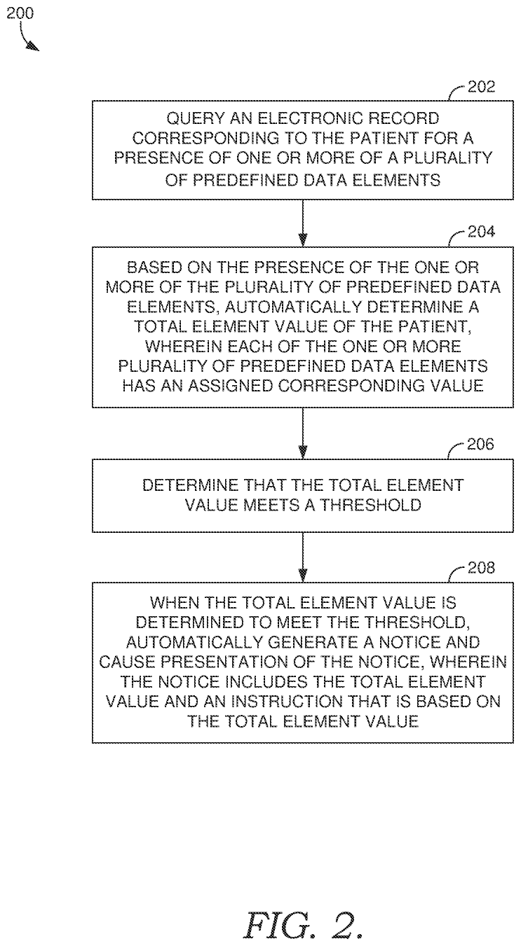

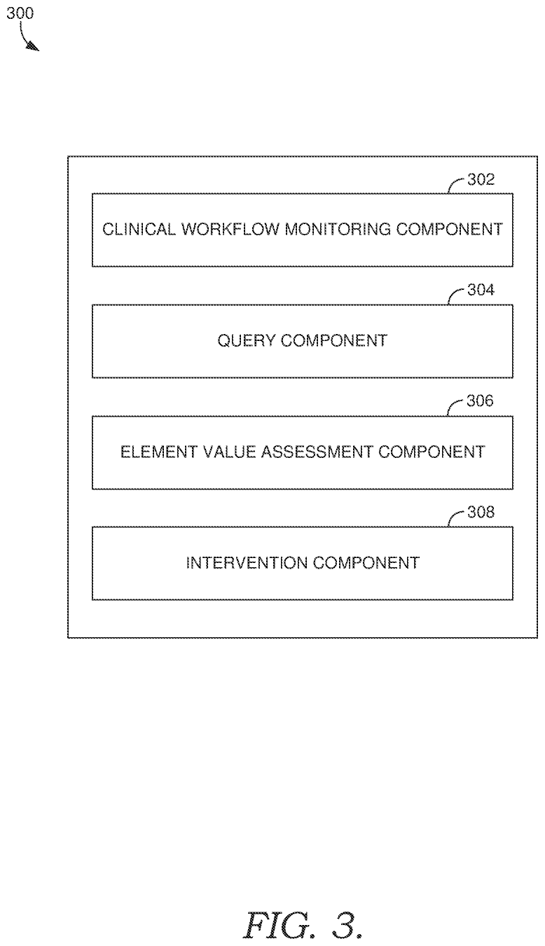

Turning now to FIGS. 2 and 3, methods are discussed that can be performed via one or more of the devices, components, and/or component interactions previously described in FIG. 1. It should be understood that the methods discussed herein can be implemented or performed via the execution of non-transitory computer-readable instructions and/or executable program code portions stored on computer readable media, using one or more processors. The computer-readable program code can correspond to the application, described above, wherein the application performs the methods, in some aspects. In aspects, the methods can be implemented and performed using a computerized application. As such, the methods can be computer-implemented methods, in some aspects, integrated with and executed to complement a computerized clinical workflow.

FIG. 2 depicts a method 200 for predicting medication induced respiratory depression, in accordance with one or more aspects. Generally, one or more events are instantiated within a computerized clinical workflow and/or one or more events are logged, which can trigger the creation and start of a computerized clinical workflow for a particular patient. For example, an admission event can be electronically instantiated, wherein the admission event is an electronic documentation record that encodes information as data, the information documenting the beginning of an inpatient admission of a specific patient (e.g., John Doe at Big City Hospital, date and time, medical record number, reason for admission). Once the admission event is logged in the computerized clinical workflow, for example, one or more additional events can be input and/or triggered in order to electronically encode information documenting medical orders, administration of medicine and treatment, diagnosis events, vital signs, laboratory orders, laboratory results, adverse events, surgical events, therapy events, room transfers, and other clinical events, up to and including discharge of the patient from the inpatient service. For simplicity, the method 200 is discussed with regard to a single patient, such that one patient is being monitored and their risk assessed throughout the clinical workflow from, generally, inpatient admission to inpatient discharge. It should, however, be understood that the method 200 can be performed concurrently to monitor and individually track each of a plurality of patients (e.g., scale of hundreds and thousands) such that the method 200 is not limited to performance for a single patient. As such, the method 200 can be implemented to track both individual patients and whole patient populations, in some aspects. Further, with regard to FIG. 3, one or more components of a computing device are shown that are specially configured to perform the method 200.

Beginning with block 202, for each instance that an indication is received for an admission event, a diagnosis event, a clinical event, or an order event corresponding to a patient, an electronic record corresponding to the patient is queried for the presence of one or more of a plurality of predefined data elements. In one aspect, the indication received corresponds to a clinical event, wherein the clinical events includes documentation of the administration of an opioid, benzodiazepine, and/or narcotic medication to a particular patient. Based on a determined that the clinical event corresponds to the administration of an opioid, benzodiazepine, and/or narcotic medication to a particular patient, a query of an electronic record for the particular patient is automatically triggered. In another aspect, a query is only triggered when the indication corresponds to a clinical event of an administration of the administration of an opioid, benzodiazepine, and/or narcotic medication to a patient. In some aspects, a clinical workflow monitoring component 302 of a computing device 300, as shown in FIG. 3, may monitor a clinical workflow and can receive indications of events. Additionally, a query component 304, as shown in FIG. 3, may automatically query the electronic record. An electronic record corresponding to the patient can be a longitudinal patient record encoding the patient's medical history, pre-existing conditions, acute and chronic conditions, diagnosis codes, prior surgeries, previous clinical visits (e.g., inpatient and ambulatory), prescription history and current medications, and the like.

In one example, an admission event can be electronically instantiated, wherein the admission event is an electronic record that encodes information as data. The encoded information may document the beginning of an inpatient admission of a specific patient (e.g., John Doe at a Big City Hospital on date and time, reasons for admission, inpatient service). Concurrently with the admission event, a diagnosis event can be electronically instantiated, wherein the diagnosis event is an electronic record that encodes information as data. The diagnosis information may include one or more specified existing diagnoses of the specific patient (e.g., John Doe has acute and/or chronic medical conditions such as respiratory distress and heart disease, respectively). Subsequent or concurrently with the admission event, an order event can be electronically instantiated, in aspects. The admission event is an electronic record that encodes information as data, and the information documents the ordering of one or more laboratory tests to be performed for the particular patient (e.g., request ordering a blood panel, urinalysis, and chest X-ray for the patient), in some aspects. Additionally or alternatively, in various aspects, a clinical workflow that corresponds to the patient is queried, wherein the clinical workflow is a computerized event log and documentation software for tracking and monitoring patient states, admission events, diagnosis events, order events, clinical events, surgical events, therapeutic agent administrations, therapeutic treatments, therapies, and the like.

An indication received for any one of an admission event, a diagnosis event, a clinical event, or an order event may be used to initiate or trigger the query. Generally, an admission event refers to an electronically instantiated instance in a computerized clinical workflow that documents the admission of a patient to a service at a healthcare facility. In some aspects, a diagnosis event refers an electronically instantiated instance in a computerized clinical workflow that documents one or more diagnoses of a patient, whether acute, chronic, pre-existing, or the like. An order event refers to an electronically instantiated instance in a computerized clinical workflow that documents an input request for one or more laboratory tests, treatment services (e.g., physical therapy), and/or medical administration of one or more therapeutic agents (e.g., pharmacological drugs, intravenous infusions, oxygen), in various aspects. A clinical event refers to an electronically instantiated instance that documents a clinical event other than an admission, diagnosis, or order. For example, a clinical event could document a patient's allergic reaction to a medication that is administered, a seizure, or a coding patient state.

For each indication of an admission event, a diagnosis event, a clinical event (e.g., an administration of one or more opioid, benzodiazepine, or narcotic medicines), or an order event corresponding to the patient (e.g., an order input for the administration of one or more opioid, benzodiazepine, or narcotic medicines), a separate query is performed to search for the one or more of a plurality of predefined data elements. However, in some aspects, when multiple events (i.e., any combination of two or more of admission event, diagnosis event, clinical event, or order event corresponding to the one patient) are simultaneously or concurrently input, instantiated, and/or received as indications, a single query can be performed in order to conserve computer processing resources. Limiting concurrent orders to triggering one query can be beneficial for computer processing conservation when the method 200 is deployed to monitor and track a plurality of patients. In contrast, when multiple events (i.e., any combination of admission event, diagnosis event, clinical event, or order event corresponding to one patient) are input, instantiated, and/or indications are received at different times on the same date, or on different dates, a separate query can be performed for each non-concurrent indication of an admission event, a diagnosis event, a clinical event, or an order event for the patient. Accordingly, queries can be performed multiple times throughout a clinical workflow for a specific patient, which, as further illustrated by the method 200 herein, provides an up-to-date risk assessment at different dates and times for a patient that accurately reflects the patient's current state.

The data elements that are searched can generally correspond to one or more items that are associated with risk for over sedation and/or medication induced respiratory depression. The electronic patient record and/or a patient-specific clinical workflow can be queried to scan and search both structured data (e.g., data formatted into fields, HL7 messages and protocol) and unstructured data (e.g., recognizing that an image is an X-ray, MRI, or video, identification of an EEG or EKG waveforms) when searching for data elements. The query is performed by concurrently searching for multiple predefined data elements, in some aspects, meaning that the plurality of data elements are all being searched for together. Examples of data elements and items that correspond to data elements include: a body mass index that is equal to or greater than 30; a result indicating renal impairment; a surgical event with anesthesia occurring within the previous 24 hours; an order for or administration of an opioid antagonist within the previous 24 hours; an order for or administration of a sedative; an order for or administration of an opioid; or an order for or administration of three or more liters of oxygen. Further examples of data elements include: an ICD-10 code for one or more of pulmonary disease, heart failure, or sleep apnea, and/or a SNOMED code for one or more of pulmonary disease, heart failure, or sleep apnea.

Further, the query can concurrently search for items such as the patient's height and weight (e.g., admission event or patient history), and the query can determine whether a data element (e.g., BMI that is greater than or equal to 30) is present by performing its own calculations to determine the patient's BMI, in one example. The query can search for a renal function impairment item by locating a specific creatinine laboratory result (e.g., results event), wherein the query can determine whether the patient's value specified in the creatinine laboratory result is greater than or equal to a predetermined value (e.g., value defined as of 1.5 milligrams per deciliter) that is utilized as indicating renal function impairment, in another example. When the creatinine laboratory result is greater than or equal to the predetermined value, the query can determine that the data element of “renal impairment” is present in the patient's record. In another example, the query can search for an item such as a surgical recovery patient “out” time (i.e., a clinical event with a date and time that specifies when a patient is moved from the operating room to a recovery location, such as a recovery floor, area, room, or bay in a post-anesthesia care unit (PACU)), and the query can perform its own calculations to determine whether a data element is present for a surgical event with anesthesia, by using the current date and time relative to the recovery patient “out” time. In yet another example, the query can search for items that include opioid prescriptions, and the query can perform its determination as to whether an opioid prescription that is present in the electronic record has a morphine milligram equivalent of 60 (i.e., 60 MME). When the query does not locate an opioid prescription or an existing opioid prescription is less than 60 MME, the query can determine the patient is positive for the data element of “opioid naïve.” Additional examples of data elements include age ranges, such as a first age range of 60 to 69 years, a second age range of 70 to 79 years, and a third age range of 80 years or greater. As such, the query can search for items such as the patient's date of birth, and the query can perform its own calculation using the present date and time in order to determine the patient's current age and to determine when the patient's age corresponds to any of the age range data elements.

Accordingly, the query discussed herein includes a search function that has processing and contextual intelligence. For example, the query can search, perform calculation, and make data element determinations by leveraging contextual information of the patient (date or birth, date and time, etc.) and other items located by the query within the electronic patient record and/or patient-specific clinical workflow. The query can simultaneously or concurrently search for any and all of the data elements discussed, and can concurrently make determinations in real-time with the search as described. In some aspects, the query only searches the clinical workflow and/or electronic record back in time to the most recent inpatient admission event.

At block 204, based on the presence of the one or more of the plurality of predefined data elements, a total element value of the patient is automatically determined, wherein each of the one or more plurality of predefined data elements has an assigned corresponding value. In some aspects, an element value assessment component 306, as shown in FIG. 3, may automatically determine the total element value of the patient. In one example, for each of the data elements that were located and/or determined to be present based on the search by the query function, the corresponding data in the electronic patient record and/or patient-specific clinical workflow can be flagged. Using the flagged data corresponding to “present” data elements, the total element value for the patient can be calculated. For example, for each data element that is present based on the query, an index of predefined values is referenced. Each data element can have a predefined corresponding value in the index. As such, the predefined values for each data element that was present is retrieved and can be summed together to calculate the total element value of the patient, as dynamically assessed at that time, based on the query. Further, the total element value of a specific patient can be dynamically re-assessed at each instance when a query is triggered, by summing together the assigned values (retrieved from the index) of each of the plurality of predetermined data elements that were located in at least one of the electronic patient record and/or the clinical workflow via the query. As the presence of data elements may change over time in the patient's record and workflow, the total element value may also change for the patient. In this manner, the total element value is dynamically determined each time that a query is triggered, thereby providing an up-to-date, accurate representation of the patient's present state with regard to predicted medication induced over-sedation.

In one illustrative example, the query component 304 can determine that one or more events in the patient-specific clinical workflow and/or in the electronic record include data elements such as: a body mass index that is equal to or greater than 30; a surgical event with anesthesia occurring within the previous 24 hours (e.g., relative to the time the query is run); an order request and/or administration of an opioid antagonist within the previous 24 hours; an order request and/or administration of a sedative occurring; a first age range of 60 to 69 years, and an ICD-10 code for pulmonary disease. In this example, the index of predefined values can be referenced by the element value assessment component 306, and values for each of the body mass index that is equal to or greater than 30, the surgical event with anesthesia occurring within the previous 24 hours, the order request and/or administration of an opioid antagonist within the previous 24 hours, the order request and/or administration of a sedative occurring within the first age range of 60 to 69 years, and the ICD-10 code for pulmonary disease can be identified by matching the data elements to the values, and/or by matching the determinations of the query component 304 to values. These values can be summed together by the element value assessment component 306 to calculate the patient's total element value, as dynamically assessed at that particular point in the workflow/date and time.

In another example, the query component 304 can determine that one or more events in the patient-specific clinical workflow and/or in the electronic record include data elements such as: a result indicating renal impairment, an order for or administration of an opioid; or an order for or administration of three or more liters of oxygen, and a third age range of 80 years or greater. In this example, the total element value can be determined, by the element value assessment component 306, to be 16. The total element value is reached by summing the corresponding assigned value of 2 for the result indicating renal impairment, the corresponding assigned value of 4 for the order for or administration of an opioid, the corresponding assigned value of 4 for the order for or administration of three or more liters of oxygen, and the corresponding assigned value of 6 for the third age range of 80 years or greater, as referenced in the index.

In yet another example, the query component 304 can determine that one or more events in the patient-specific clinical workflow and/or in the electronic record include data elements such as: one or more SNOMED codes for sleep apnea and a body mass index equal to or greater than 30. In this example, the element value assessment component 306 can determine that the total element value is 2, based on the corresponding assigned value of 1 for the SNOMED code for sleep apnea, and the corresponding assigned value of 1 for a body mass index that is equal to or greater than 30, as referenced in the index.

In one example, the query component 304 can determine that one or more events in the patient-specific clinical workflow and/or in the electronic record include items/data elements such as: concurrent prescriptions, orders, or administrations of a sedative and an opioid (e.g., drugs of the class benzodiazepine and drugs of the class opioid). In this example, the element value assessment component 306 can determine that the total element value is 2, wherein the concurrent prescriptions, orders, or administrations of a sedative and an opioid is a data element having a value of 2, as referenced in the index. As such, the query component 304 can search for individual data elements and combinations of data elements, where the combinations themselves have an assigned corresponding value in the index.

It should be understood that the values in the index can be any numerical value or fraction, and are not limited to whole numbers, which are used herein merely as an example for simplicity. Further, it should be understood that one or more values in the index can correspond to specific combinations of data elements, where the single data elements occurring alone correspond to a null or value of zero, in some aspects. As such, some individual data elements may contribute to the patient's total element value when located in combination with other data elements. In some aspect, for example, a data element can have a first value in the index that is assigned to the data element occurring alone (e.g., one event of oxygen administration equal to or greater than 3 liters), a second value that is assigned to the data element occurring more than once (e.g., three separate events of oxygen administrations equal to or greater than 3 liters), and a third value that is assigned to the data element in a specific combination with one or more other data elements, (e.g., an event for oxygen administration equal to or greater than 3 liters and an event for administration of naloxone). As such, some data elements can be searched individually and searched in combination with other data elements by the query function. Additionally, it should be understood that one or more data elements queried can have a corresponding negative value (e.g., −2) stored in the index, such that when summed with other positive values for other located data elements, the total element value for a patient can be lowered. For example, data elements that are associated with decreasing a patient's risk for over-sedation may be assigned a negative numerical value in the index.

Continuing to block 206, the method 200 determines whether the total element value meets a threshold. In some aspects, the element value assessment component 306 can determine whether the element value meets a threshold. The threshold can be a predetermined value or a predetermined value range (e.g., values 3 to 6, value of 7 or greater). As discussed later herein, the total element value can be assessed relative to a plurality of different thresholds. Generally, the threshold is a value that indicates a clinically non-negligible risk level for medication induced respiratory depression (e.g., opioid induced). As such, when a threshold is met, the method 200 predicts that the patient is at risk for over-sedation/medication induced respiratory depression.

At block 208, when the total element value is determined to meet the threshold, a notice is automatically generated and the notice is automatically communicated for presentation, for example, via a graphical user interface. In some aspects, an intervention component 308 can generate and communicate the notice for presentation. The notice includes a warning of potential over sedation of the patient, in some aspects. For example, the notice indicates that the specific patient is at risk for medication induced respiratory depression based on the total element values that are determined dynamically in real-time, in response to the query that was triggered by indication(s) of an admission, diagnosis, order, and/or clinical event.

The notice can include the total element value, in some aspects. In further aspects, the notice itself and/or as displayed includes an instruction to perform an intervention that is based on the total element value. The instruction for intervention can be automatically selected, by the intervention component 308 of FIG. 3, and provided with the notice in response to determining that a threshold is met by the patient's total element value. In some aspects, the intervention component 308 can identify, select, or generate a particular instruction, and can send the instruction with or as a part of the notice. For example, the instruction can specify a medical order that requests the administration of pulse oxygen every n of hours and monitoring of vital signs for the patient. In another example, the instruction can specify a medical order that requests the administration of continuous pulse oxygen for the patient. In yet another example, the instruction can specify a medical order that requests an end tidal CO2 (EtCO2) protocol for the patient.

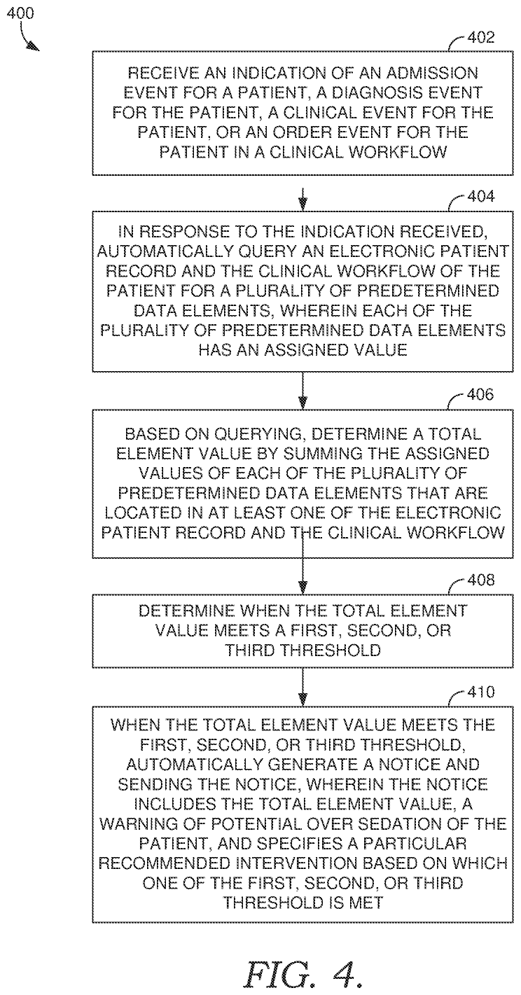

Turning now to FIG. 4, another method 400 is depicted for predicting medication induced respiratory depression, in accordance with aspects. In some aspects, the method 400 can be performed, using one or more processors to execute computer-readable or machine instructions stored or embodied on a non-transitory computer-readable storage medium. As some aspects of the method 400 are previously described with regard to other methods herein, those aspects are only discussed briefly.

Beginning with block 402, an indication of an admission event, a diagnosis event, a clinical event, or an order event is received for a particular patient in a clinical workflow. In some aspects, any admission event, diagnosis event, clinical event, or order event can trigger a query for data elements. In an aspects, the plurality of data elements include a plurality of diagnosis codes that are specific to at least one of pulmonary disease, heart failure, or sleep apnea. Additionally or alternatively, admission events, diagnosis events, clinical events, or order events that contain specific information or particular clinical data can be used to particularly trigger a query for data elements. In one further example, a query can be triggered only when the admission event, the diagnosis event, the clinical event, or the order event is determined to include or encode one or more items that correspond to the plurality of data elements, including: a surgical event with anesthesia occurring within previous 24 hours; an order for or administration of an opioid antagonist within previous 24 hours; an order for or administration of a sedative; an order for or administration of an opioid; and/or an order for or administration of three or more liters of oxygen.

At block 404, in response to the indication received, an electronic patient record and the clinical workflow of the patient is queried for a plurality of predetermined data elements. In one aspect, the indication received corresponds to a clinical event, and the clinical events includes documentation of an administration of an opioid, benzodiazepine, and/or narcotic medication to a particular patient. Based on a determination or recognition that the clinical event corresponds to the administration of an opioid, benzodiazepine, and/or narcotic medication to a particular patient, the query of an electronic record for the particular patient is automatically triggered. Each of the plurality of predetermined data elements has an assigned value, whether the same, similar, or different, for example, which can be stored in an index. For example, one or more data elements that are strongly associated with adverse outcomes (e.g., medication induced respiratory depression) can be assigned a higher value than other data elements that are less strongly associated with adverse outcomes. When querying the electronic patient record and the clinical workflow of the patient, the query may search backward in time from the current date and time up to the most recent admission event, in some aspects. The query function may search backwards in time for a defined period of time (e.g., one year from the current date and time).

At block 406, based on the query, a total element value is determined by summing the assigned values of each of the plurality of predetermined data elements that are located in at least one of the electronic patient record and the clinical workflow. As such, the predetermined values assigned to each of the data elements that were found to be present in the record(s) are retrieved from the index and summed together to calculate the overall total element value for the patient, in aspects. Generally, the total element value reflects the patient's current state with regard to risk for medication induced respiratory depression.

At block 408, the total element value is determined to meet a first, second, or third threshold, in some aspects. In aspects, the first, second, and third thresholds are predefined values to which a patient's total element value can be compared, wherein each threshold reflects an increasing risk of medication induced respiratory depression. For example, the first threshold can have a preset value of 5, the second threshold can have a preset value of 8, and the third threshold can have a preset value of 10. It should be understood that these values are merely examples, and the preset values of thresholds generally reflect the numerical scale of the values that have been assigned to data elements. In some aspects, when a third and highest threshold is met by the total element value of the patient, any further calculation of additional values for data elements can be halted (i.e., a high or maximum threshold has been met such that further assessment is redundant or additional values are cumulative in nature).

At block 410, when the total element value meets the first, second, or third threshold, a notice is automatically generated and sent, for example, to a user such as a clinician. The notice, in some aspects, includes the total element value for the patient, a warning of potential over sedation of the patient, and can also specify a particular recommended intervention that is based on which one of the first, second, or third threshold has been met. Additionally, in some aspects, the notice includes each of the plurality of predetermined data elements that were located in at least one of the electronic patient record or the clinical workflow, as well as the corresponding value of each of the plurality of predetermined data elements that were located.

In aspects, the particular recommended intervention reflects the specific threshold that has been met, wherein that threshold reflects the predicted over-sedation risk. For example, when the total element value meets the first threshold, the particular recommended intervention can specify a medical order for administration of pulse oxygen every n of hours and monitoring of vital signs for the patient. In some aspects, the medical order (e.g., administration of pulse oxygen every n of hours and monitoring of vital signs for the patient) is automatically generated with the notice and input to the clinical workflow of the patient for performance. In another example, when the total element value meets the second threshold that is greater than the first threshold, the particular recommended intervention can specify a medical order for administration of continuous pulse oxygen for the patient. In such an aspect, the medical order (e.g., administration of continuous pulse oxygen) is automatically generated with the notice and input to the clinical workflow of the patient for performance. In one example, when the total element value meets the third threshold that is greater than the first and second thresholds, the particular recommended intervention can specify performance of an end tidal CO2 (EtCO2) protocol for the patient. In an aspect, the medical order (e.g., end tidal CO2 (EtCO2) protocol) is automatically generated with the notice and input to the clinical workflow of the patient for performance.

As such, the recommended intervention can include a specific clinical instruction to perform a detailed interventional task in response to the particular threshold that has been met. Accordingly, in some aspects, as patient risk for medication induced respiratory depression increases and a higher threshold is met by that patient's total element value, the particular recommended intervention more aggressively treats the patient who is at risk. Additionally, in some aspects, it can be determined whether the particular recommended intervention that was sent with the notice has not been performed within a predetermined period of time relative to the date and time the notice was generated and/or sent (e.g., based on an absence of documentation for the particular recommended intervention being logged in the clinical workflow for the patient). In response, a second subsequent notice can be automatically generated and sent to a clinician requesting completion or confirmation of completion of the particular recommended intervention.

In some aspects, the method 400 can be iteratively performed when queries are triggered by particular event indications in the clinical workflow of a patient. As an example, a subsequent indication is received for a diagnosis event, clinical event, or order event for the patient. In response to the subsequent indication received, in aspects, the electronic patient record and the clinical workflow of the patient are again automatically queried to search for and locate one or more of the plurality of predetermined data elements, in the example. Then, based on the subsequent query, a new total element value of the patient is determined by summing the assigned values of each of the plurality of predetermined data elements that are located in at least one of the electronic patient record and the clinical workflow, for example. When the new total element value meets the first, second, or third threshold, a new notice can be automatically generated and sent to a clinician and/or input to the clinical workflow, wherein the notice includes the new total element value and the warning of potential over sedation of the patient, in this example. Accordingly, a plurality of notices and orders for clinical intervention(s) for the specific patient can be automatically generated and populated into a clinical workflow for the patient based on the dynamic total element value determinations made throughout the clinical workflow.

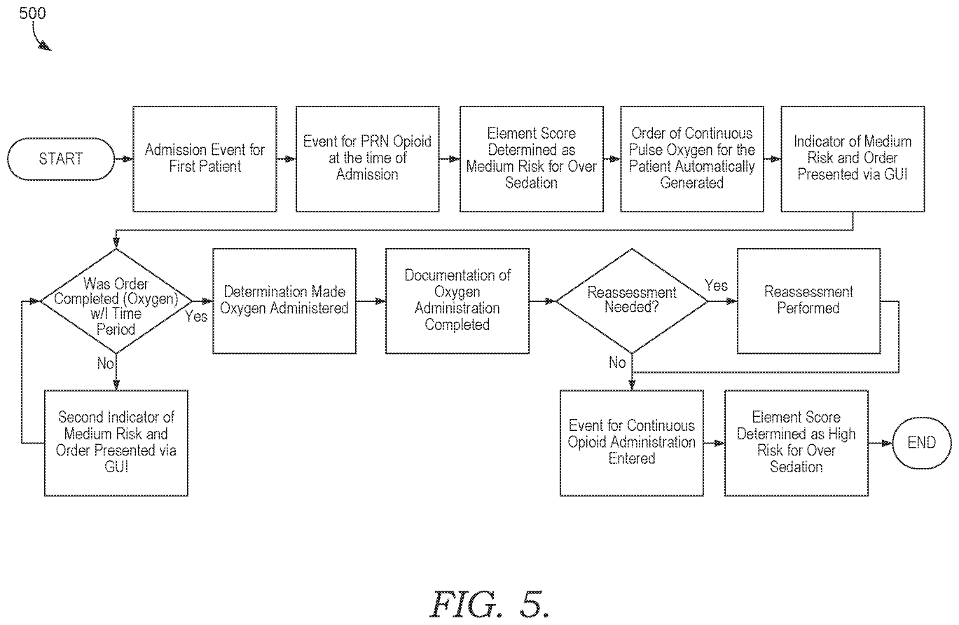

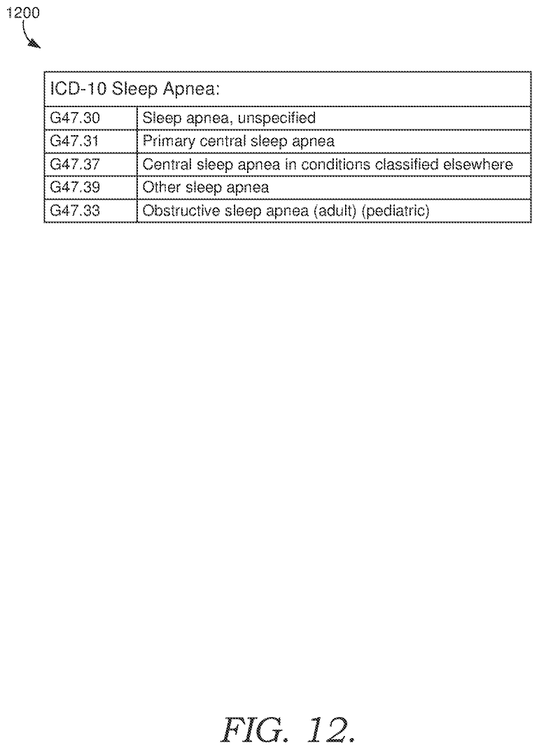

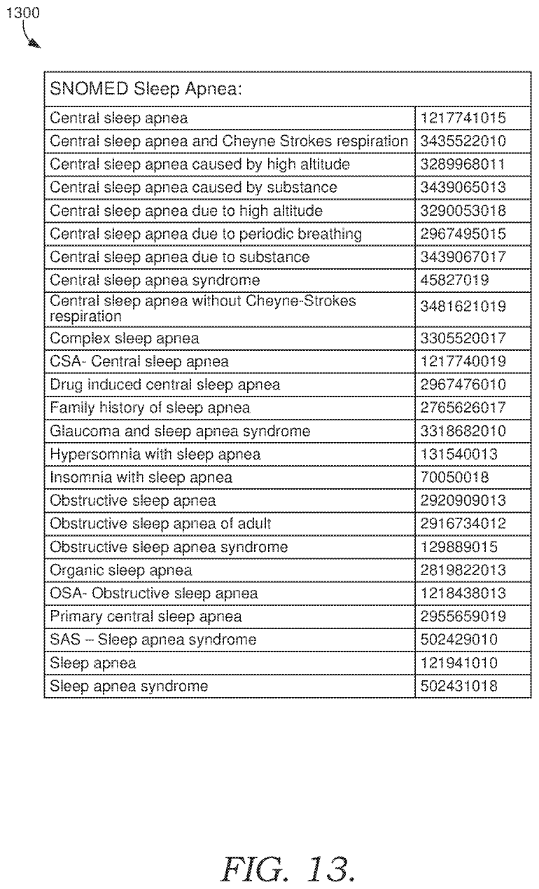

Continuing to FIGS. 5 through 13, illustrative workflows, example implementations, and graphical user interfaces are depicted with regard to the previously described aspects. As such, these figures are discussed briefly, though it will be understood that the previous discussion and details described therein can be applicable to FIGS. 5 through 13. FIG. 5 illustrates a flowchart of an example implementation that predicts medication induced respiratory depression. In the example of FIG. 5, an indication is received that specifies a first patient is being admitted to an inpatient service. The indication, for example, corresponds to an admission event entered or input to a clinical workflow in order to document the admission of the patient, for example. In the example of FIG. 5, an indication of an order event is received in the clinical workflow for the patient. The order event in this example includes a medication order specifying administration of an opioid PRN for the patient being admitted. In response to the admission event and/or the order event, the electronic patient record and the clinical workflow of the patient are automatically queried to locate any of a plurality of predetermined data elements. Based on the particular predetermined data elements that are found using the query, a total element value is automatically generated by summing the assigned values of each of the plurality of predetermined data elements that are located in the electronic patient record and/or the clinical workflow for the patient. When the total element value meets a first threshold (e.g., when the total element value meets a threshold of 6), a new order event is automatically generated and added to the clinical workflow of the patient. For example, a medication order specifying administration of pulse oxygen for the patient is automatically generated based on the determination that the patient has a medium risk of over sedation, i.e., when the total element value meets a threshold corresponding to a medium risk, as shown in FIG. 5. Additionally, a notice is generated and presented to a clinician to report the patient's medium risk level of medication induced respiratory depression in response to determining that the total element value meets the threshold. Accordingly, based on total element value determinations relative to one or more thresholds, one or more notices, alerts, alarms, messages, and/or medical orders can be automatically generated and input to a clinical workflow. In this manner, the total element value determination for a patient can be determined, updated, and/or reassessed at multiple points throughout the patient's inpatient treatment cycle.

In the example of FIG. 5, subsequent to the input of the continuous oxygen medical order to the clinical workflow for the patient, a timer can be initiated and/or a workflow checkpoint can be used to determine whether the continuous oxygen medical order has been administered to the patient based on the presence or absence of clinician documentation of performance. When the clinical workflow for the patient lacks documentation indicating that the medical order has been performed and/or administration completed, a second subsequent notice, alert, alarm, or message can be automatically generated and communicated to a clinician, in aspects. The second subsequent notice acts as a reminder that the medical order for the intervention is recommended and/or needed to reduce or otherwise prevent an increase in the patient's risk for medication induced respiratory depression. In the example, the second subsequent notice can be presented in a graphical user interface. In an instance when the clinical workflow for the patient includes a determination and/or documentation indicating that the medical order has been performed and/or administration is completed, as shown in FIG. 5, the total element value of the patient can be determined again (i.e., reassessment can be performed).

Further, in the example of FIG. 5, a third indication is received for an order event specifying a continuous opioid administration for the patient. In response to the third indication (i.e., the new order event), the electronic patient record and the clinical workflow of the patient are again automatically queried to locate any of a plurality of predetermined data elements and identify the particular assigned value of each element located, using the admission event as the starting “time point” for searching to the present date and time, which encompasses the new order event that triggered the query. Based on the predetermined data elements that are found using the query, an updated or new total element value is automatically generated by summing the assigned values of each of the plurality of predetermined data elements that are located in the electronic patient record and/or the clinical workflow for the patient. When the total element value meets the first, second, or even a third threshold (e.g., when the total element value meets a threshold of 8), the patient can be assessed as a high risk for medication induced over sedation. As such, an order specifying another recommended clinical intervention for the patient can be automatically generated and populated, without user input, to the clinical workflow for the patient (not shown). The patient's risk can be reassessed multiple times from admission through the patient's discharge from the inpatient service, in aspects.

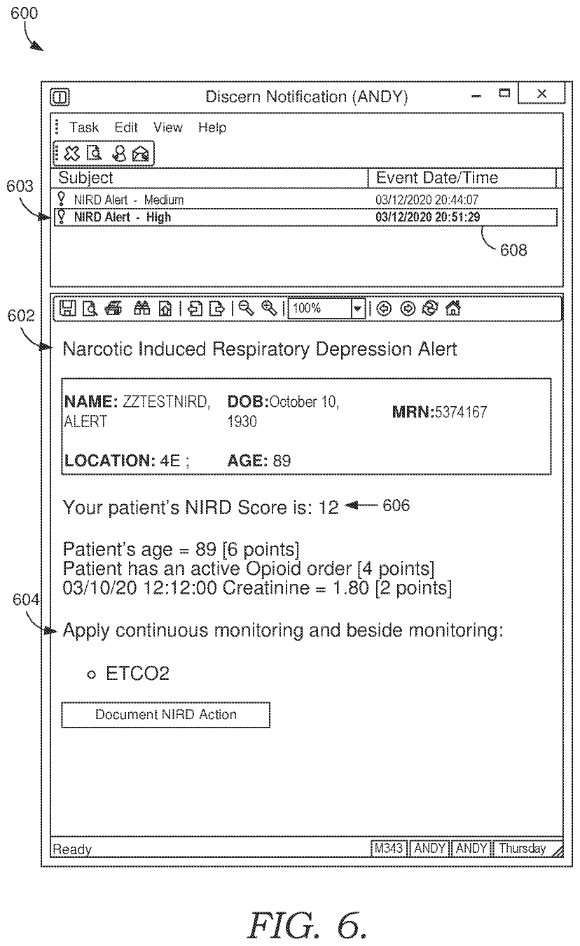

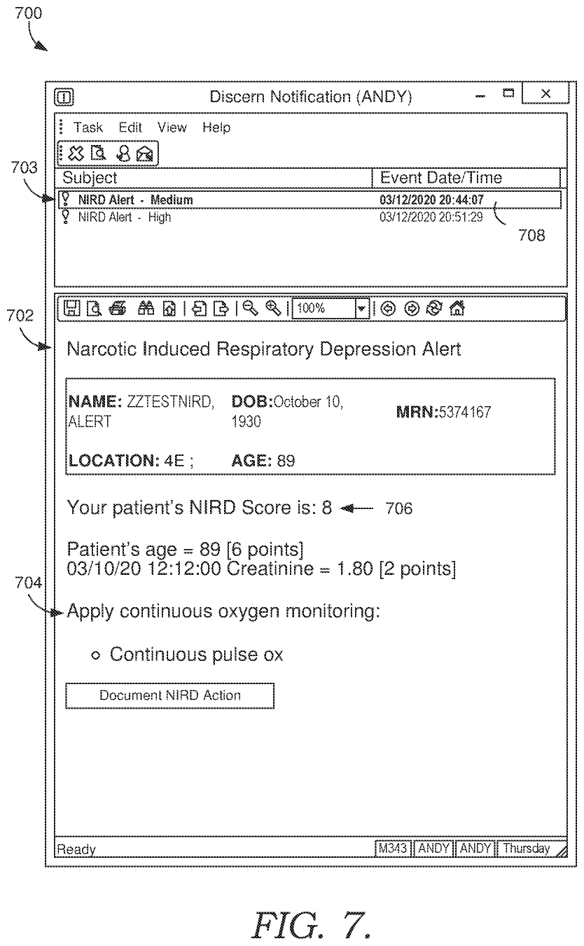

FIGS. 6 and 7 depict example graphical user interfaces for displaying predictions of medication induced respiratory depression, in accordance with aspects. FIGS. 6 and 7 include graphical user interfaces 600 and 700 that include notices 602 and 702. For example, the notices 602 and 702 can include one or more messages indicating a high risk level 603 or a medium risk level 703 for a particular patient. Additionally, the notices 602 and 702 can provide patient identification (e.g., patient name, age, medical record identifier, room, date of birth), the total element value for that patient, and a recommended intervention. For example, a particular instruction or recommendation 604 can be provided in response to the determination of the high risk level 603, wherein the high risk level 603 is based on a specific threshold being met by the patient's total element value 606. In another example, a particular instruction or recommendation 704 can be provided in response to the determination of the medium risk level 703, where the medium risk level 703 is based on another, lower threshold being met by the patient's total element value 706. The particular time 608 and 708 of the notices can be displayed in the notices 602 and 702 as well, which allows a clinician to view the progression of a patient's risk level for medication induced respiratory depression.

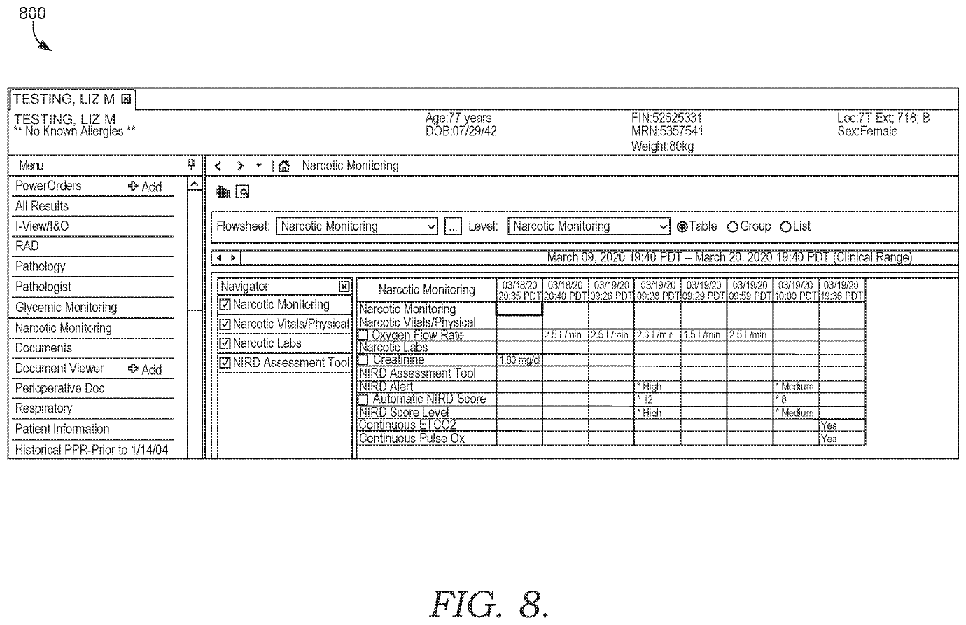

FIG. 8 depicts an example graphical user interface 800 for displaying predictions of medication induced respiratory depression, in accordance with aspects. In this example, one or more notices for a single patient are concurrently presented in the same graphical user interface 800, each including a date and time for each notice, the level (e.g., high, medium) met by the patient's total element value for which the notice was communicated, and a total data element value of the patient (e.g., 12, 8) corresponding to the level/notice. Additionally, documentation and/or completion of one or more order events can be presented concurrently with the notice information (e.g., Continuous EtCO2 indication “Yes” along with a date and time for the administration of the continuous EtCO2). In this manner, the graphical user interface 800 presents a single patient-specific timeline of assessments, risk level determinations, data element value totals, intervention(s) administered, and corresponding dates and times in the clinical workflow for medication inducted respiratory depression risk of a particular patient.

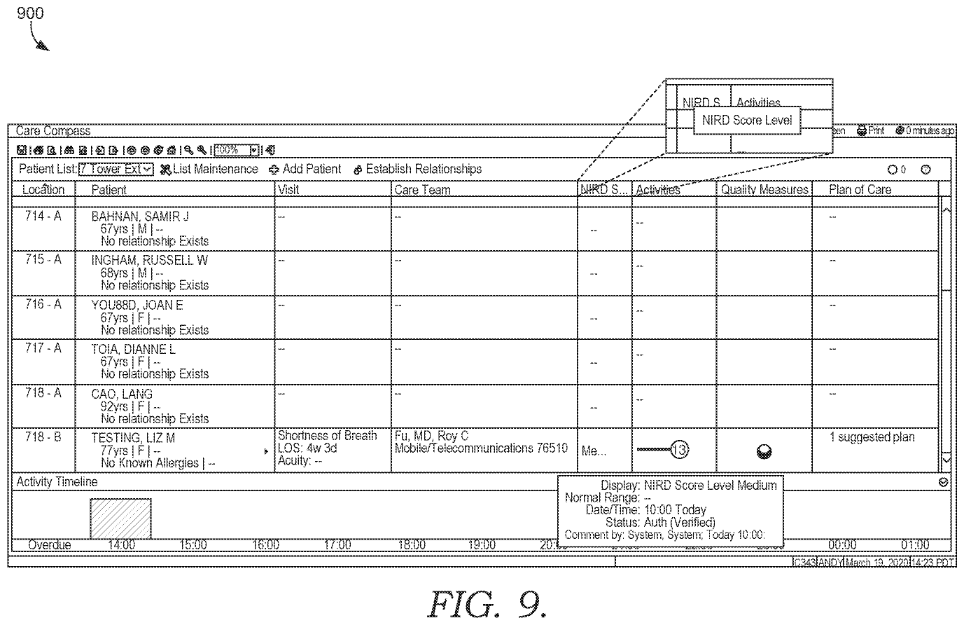

FIG. 9 depicts an example graphical user interface 900 for displaying predictions of medication induced respiratory depression, in accordance with aspects. The graphical user interface 900 can concurrently present a notice of risk level determinations for a plurality of patients and/or instructions for intervention, for example, using one or more rows and columns.

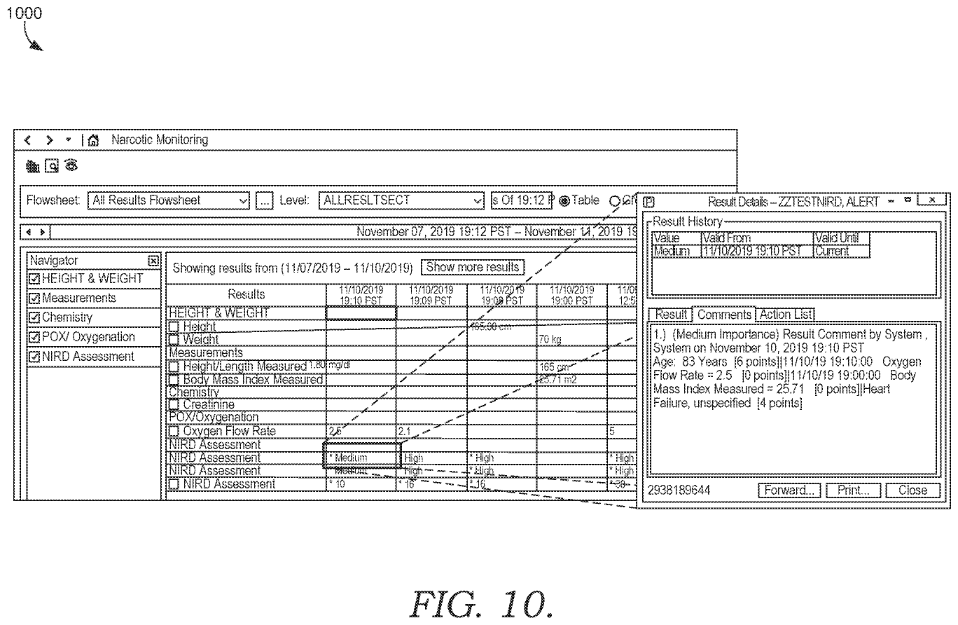

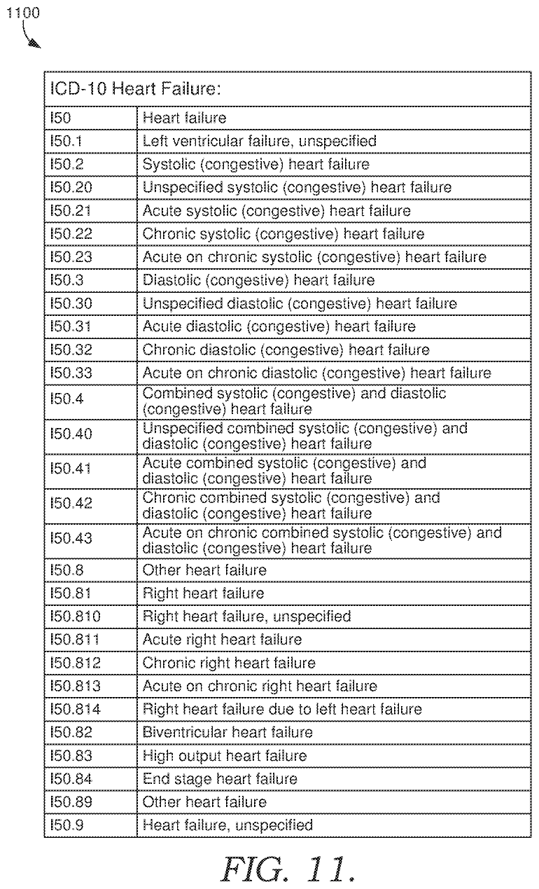

FIG. 10 depicts an example graphical user interface 1000 for displaying predictions of medication induced respiratory depression, in accordance with aspects. The graphical user interface 1000 can concurrently present, as one or more user selectable elements 1002, one or more notices of risk level determinations, total element values, and the like. For example, by selecting one of the one or more user selectable elements 1002 in the graphical user interface 1000, a popup window 1004 can be presented that specifies details such as the total element value, each of the data elements that were identified as present in the record by the query, and the assigned values for each of the data elements located in the query and which contributed to the total element value. FIGS. 11-13 include tables of examples of medical codes for heart failure and sleep apnea which may be queried as data elements, in accordance with aspects discussed herein.

The present invention has now been described in relation to particular aspects, which are intended in all respects to be illustrative rather than restrictive. Thus the present invention is not limited to these aspects, but variations and modifications can be made without departing from the scope of the present invention.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A method for predicting medication induced respiratory depression, the method comprising:
for each instance of receipt of an indication of an admission event, a diagnosis event, a clinical event, or an order event corresponding to a patient:
querying an electronic record corresponding to the patient for a presence of one or more of a plurality of predefined data elements;
based on the presence of the one or more of the plurality of predefined data elements, automatically determining a total element value of the patient, wherein each of the one or more plurality of predefined data elements has an assigned corresponding value;
determining that the total element value meets a threshold; and
when the total element value is determined to meet the threshold, automatically generating a notice and causing presentation of the notice, wherein the notice includes the total element value and an instruction that is based on the total element value.

2. The method of claim 1, wherein the plurality of predefined data elements correspond to data that encodes one or more of:
an ICD-10 code for one or more of pulmonary disease, heart failure, or sleep apnea; or
a SNOMED code for one or more of pulmonary disease, heart failure, or sleep apnea.

3. The method of claim 1, wherein the plurality of predefined data elements correspond to data that encodes one or more of:
a body mass index equal to or greater than 30;
a result indicating renal impairment;
a surgical event with anesthesia occurring within previous 24 hours;
an order for or administration of an opioid antagonist within previous 24 hours;
an order for or administration of a sedative;
an order for or administration of an opioid; or
an order for or administration of three or more liters of oxygen.

4. The method of claim 1, wherein the plurality of predefined data elements correspond to a first age range of 60 to 69 years, a second age range of 70 to 79 years, or a third age range of 80 years or greater.

5. The method of claim 1, wherein the notice includes a warning of potential over sedation of the patient.

6. The method of claim 1, wherein the instruction specifies a medical order for administration of pulse oxygen every n of hours and monitoring of vital signs for the patient.

7. The method of claim 1, wherein the instruction specifies a medical order for administration of continuous pulse oxygen for the patient.

8. The method of claim 1, wherein the instruction specifies performance of an end tidal CO₂ (EtCO2) protocol for the patient.

9. A non-transitory computer-readable storage medium having instructions embodied thereon, the instructions being executable by one or more processors to perform a method for predicting medication induced respiratory depression, the method comprising:
receiving an indication of an admission event for a patient, a diagnosis event for the patient, a clinical event for the patient, or an order event for the patient in a clinical workflow;
in response to the indication received, automatically querying an electronic patient record and the clinical workflow of the patient for a plurality of predetermined data elements, wherein each of the plurality of predetermined data elements has an assigned value;
based on querying, determining a total element value by summing the assigned values of each of the plurality of predetermined data elements that are located in at least one of the electronic patient record and the clinical workflow;
determining when the total element value meets a first, second, or third threshold; and
when the total element value meets the first, second, or third threshold, automatically generating a notice and sending the notice, wherein the notice includes the total element value and a warning of potential over sedation of the patient.

10. The media of claim 9, wherein the notice includes each of the plurality of predetermined data elements that were located in at least one of the electronic patient record or the clinical workflow, and the corresponding assigned value of each of the plurality of predetermined data elements that were located.

11. The media of claim 9, wherein the plurality of predetermined data elements include a plurality of diagnosis codes that are specific to at least one of pulmonary disease, heart failure, or sleep apnea.

12. The media of claim 9, wherein when the total element value meets the first threshold, the notice includes a particular recommended intervention specifies a medical order for administration of pulse oxygen every n of hours and monitoring of vital signs for the patient.

13. The media of claim 9, wherein when the total element value meets the second threshold that is greater than the first threshold, the notice includes a particular recommended intervention specifies a medical order for administration of continuous pulse oxygen for the patient.

14. The media of claim 9, wherein when the total element value meets the third threshold that is greater than the first and second threshold, the notice includes a particular recommended intervention specifies performance of an end tidal CO₂ (EtCO2) protocol for the patient.

15. The media of claim 9, wherein the notice includes a particular recommended intervention, and wherein the instructions being executable by one or more processors further comprise:
determining that the particular recommended intervention is not performed within a predetermined period of time from the sending of the notice based on an absence of documentation for the particular recommended intervention being logged in the clinical workflow for the patient; and
automatically sending another notice to a clinician to complete the particular recommended intervention.

16. The media of claim 9, the instructions being executable by one or more processors further comprising, determining whether the admission event, the diagnosis event, the clinical event, or the order event include one or more items that correspond to at least one of the plurality of predetermined data elements:
a surgical event with anesthesia occurring within previous 24 hours;
an order for or administration of an opioid antagonist within previous 24 hours;
an order for or administration of a sedative;
an order for or administration of an opioid; or
an order for or administration of three or more liters of oxygen.

17. The media of claim 16, the instructions being executable by one or more processors further comprising, when the admission event, the diagnosis event, the clinical event for the patient, or the order event includes at least one of the one or more of the items, triggering the automatic querying.

18. The media of claim 9, wherein the electronic patient record and the clinical workflow of the patient are automatically queried from a most recent admission event to present time and date.

19. The media of claim 9, the instructions being executable by one or more processors further comprising:
receiving a subsequent indication of another admission event, diagnosis event, clinical event, or order event for the patient;
in response to the subsequent indication received, automatically querying the electronic patient record and the clinical workflow of the patient for the plurality of predetermined data elements;
determining a new total element value by summing the assigned values of each of the plurality of predetermined data elements that are located in at least one of the electronic patient record and the clinical workflow;
determining when the new total element value meets the first, second, or third threshold; and
when the new total element value meets the first, second, or third threshold, automatically generating a new notice and sending the new notice, wherein the new notice includes the new total element value and the warning of potential over sedation of the patient.

20. A non-transitory computer-readable storage medium having instructions embodied thereon, the instructions being executable by one or more processors to perform a method for predicting medication induced respiratory depression, the method comprising:
for each instance of receipt of an indication of an admission event, a diagnosis event, a clinical event, or an order event corresponding to a patient:
querying an electronic record corresponding to the patient for a presence of one or more of a plurality of predefined data elements;
based on the presence of the one or more of the plurality of predefined data elements, automatically determining a total element value of the patient, wherein each of the one or more plurality of predefined data elements has an assigned corresponding value;
determining that the total element value meets a threshold; and
when the total element value is determined to meet the threshold, automatically generating a notice and causing presentation of the notice, wherein the notice includes the total element value and an instruction that is based on the total element value.
